(19) United States
(12) Patent Application Publication
(10) Pub. No.: US 2011/0110818 A1
(43) Pub. Date: May 12, 2011

(54) DRY SANITIZING PATCH FOR DECREASING HUMAN PATHOGEN TRANSMISSION

(76) Inventors: Melissa Mowbray-d'Arbela, Hong Kong (HK); Ranjan Marwah, Hongkong (HK)

(21) Appl. No.: 13/001,039

(22) PCT Filed: Jun. 25, 2009
(86) PCT No.: PCT/US09/48706

§ 371 Date: Dec. 22, 2010

Related U.S. Application Data
(60) Provisional application No. 61/180,085, filed on May 20, 2009.

(30) Foreign Application Priority Data
Jun. 25, 2008 (US) PCT/US2008/068225

(51) Int. Cl. A61L 2/23; A01N 25/24; A01P 1/00; A01N 59/20; B32B 37/12

(52) U.S. Cl. 422/28; 424/407; 424/631; 156/60

(57) ABSTRACT

A dry sanitizing patch for use in decreasing the transmission of one or more than one human pathogen, the dry sanitizing patch comprising: a) a binding layer comprising a fabric comprising one or more than one binding substance comprising one or more than one human pathogen binding group for chemically attaching the human pathogen to the binding substance; b) an adhesive layer connected to the binding layer, and comprising an adhesive suitable for reversibly attaching the dry sanitizing patch to a surface; and c) a backing layer removably connected to the adhesive layer to protect the adhesive layer until the patch is ready for use.

### CROSS-REFERENCE TO RELATED APPLICATIONS

The present Application claims priority from U.S. Provisional Patent Application No. 61/180,085 filed May 20, 2009 and titled “Dry Sanitizing Patch for Decreasing Human Pathogen Transmission,” and also claims priority from International Patent Application No. PCT/US2008/068225 filed Jun. 25, 2008 and titled “Devices and Methods for Decreasing Human Pathogen Transmission,” the contents of which are incorporated in this disclosure by reference in their entirety.

### BACKGROUND

There are a variety of infectious human diseases that are believed to be transmitted in part by direct skin to skin contact between individuals, or by contact of the skin of one person with a contaminated object. Among these infectious human diseases are human respiratory tract infections caused by bacteria, fungi and viruses. Examples of viral causes of infectious human diseases (and their associated diseases) include: Influenza A virus (influenza); Influenza B-C virus (coryza; ‘common cold’); Human adenovirus A-C (various respiratory tract infections; pneumonia); Human Para-influenza virus (coryza; ‘common cold;’ croup); Mumps virus (epidemic parotitis); Rubeola virus (measles); Rubella virus (German measles); Human respiratory syncytial virus (RSV) (coryza; ‘common cold’); Human coronavirus (SARS virus) (SARS); Human rhinovirus A-B (coryza; ‘common cold’); parvovirus B19 (fifth disease); variola virus (smallpox); varicella-zoster virus (herpes virus) (chickenpox); Human enterovirus (coryza; ‘common cold’); Bordetella pertussis (whooping cough); Neisseria meningitidis (meningitis); Corynebacterium diphtherias (diphtheria); Mycoplasma pneumoniae (pneumonia); Mycobacterium tuberculosis (tuberculosis); Streptococcus pyogenes/pneumoniae (strep throat, meningitis, pneumonia); and Haemophilus influenzae Type B (epiglottis, meningitis, pneumonia).

Many of the human viral respiratory tract infections result in significant morbidity and mortality. For example, seasonal epidemics of influenza viruses worldwide infect an estimated 3 million to 5 million people, and kill between 250,000 to 500,000 people each year. In addition, cyclical influenza virus pandemics occur, such as the influenza outbreak in 1918 which killed approximately 20 million people worldwide.

Frequent hand washing is recommended as one method to decrease the transmission of human pathogens, but the facilities for hand washing are often not available, and repeated hand washing can be time consuming. Additionally, it is not always possible or socially acceptable to interrupt one's activities for hand washing after potential contamination.

Therefore, there is a need for a new method for preventing transmission of one or more than one human pathogen that causes human respiratory tract infections, among other diseases.

### SUMMARY

According to one embodiment of the present invention, there is provided a dry sanitizing patch for use in decreasing the transmission of one or more than one human pathogen. In one embodiment, the dry sanitizing patch comprises: a) a binding layer comprising a material comprising three plies, and where one of the plies comprises a fabric comprising one or more than one binding substance comprising one or more than one human pathogen binding group for chemically attaching the human pathogen to the binding substance; b) an adhesive layer connected to the binding layer, and comprising an adhesive suitable for reversibly attaching the dry sanitizing patch to a surface; c) a backing layer removably connected to the adhesive layer to protect the adhesive layer until the patch is ready for use; and d) a shape defined by a perimeter; where the binding layer comprises a non-contact surface connected to the adhesive layer, and comprises an opposing contact surface for sanitizing the skin of a person or an object that is contacted with the contact surface of the binding layer; where the binding substance is CI Reactive Blue 21; where the fabric further comprises both multivalent copper and multivalent zinc; where the adhesive layer comprises a first surface connected to the non-contact surface of the binding layer and a second surface adjacent to the backing layer; where the shape defined by the perimeter is a circle with a tab extension defined only by the backing layer; and where the binding layer has a surface area defined by the perimeter of between 2 cm² and 10 cm².

According to another embodiment of the present invention, there is provided a dry sanitizing patch for use in decreasing the transmission of one or more than one human pathogen. The dry sanitizing patch comprises: a) a binding layer comprising a fabric comprising one or more than one binding substance comprising one or more than one human pathogen binding group for chemically attaching the human pathogen to the binding substance; b) an adhesive layer connected to the binding layer, and comprising an adhesive suitable for reversibly attaching the dry sanitizing patch to a surface; c) a backing layer removably connected to the adhesive layer to protect the adhesive layer until the patch is ready for use; and d) a shape defined by a perimeter; where the binding layer comprises a non-contact surface connected to the adhesive layer, and comprises an opposing contact surface for sanitizing the skin of a person or an object that is contacted with the contact surface of the binding layer; and where the adhesive layer comprises a first surface connected to the non-contact surface of the binding layer and a second surface adjacent to the backing layer. In one embodiment, the shape defined by the perimeter is selected from the group consisting of a circle, an oval, a rectangle, a square and a triangle. In another embodiment, the shape defined by the perimeter is a circle with a tab extension defined only by the backing layer. In one embodiment, the binding layer of the dry sanitizing patch has a surface area defined by the perimeter of between 2 cm² and 100 cm². In another embodiment, the binding layer of the dry sanitizing patch has a surface area defined by the perimeter of between 2 cm² and 10 cm². In another embodiment, the binding layer of the dry sanitizing patch has a surface area defined by the perimeter of between 4 cm² and 8 cm². In one embodiment, the binding substance is one or more than one reactive dye. In a preferred embodiment, the reactive dye is selected from the group consisting of CI Reactive Blue 4, CI Reactive Blue 21, CI Reactive Blue 140, CI Reactive Blue 163, CI Reactive Brown 23, CI Reactive Orange 4, CI Reactive Red 1, CI Reactive Red 2, CI Reactive Red 6, CI Reactive Red 11, CI Reactive Red 78, CI Reactive Yellow 39, and CI Reactive Yellow 86. In one embodiment, the human pathogen binding group is selected from the group consisting of a sulfate group and a sulfonate group. In one embodiment, the fabric further comprises one or more than one type of multivalent metallic ion or metallic salt. In one embodiment, the multivalent metallic ion is selected from the group consisting of multivalent copper, multivalent silver and multivalent zinc. In another embodiment, the multivalent metallic ion is selected from the group consisting of copper acetate, copper oxide, copper sulfate and zinc acetate. In one embodiment, the human pathogen binding group is selected from the group consisting of a sulfate group and a sulfonate group; and the fabric further comprises one or more than one type of multivalent metallic ion or metallic salt. In one embodiment, the binding substance comprises a linker group for attaching the binding substance to the fabric. In one embodiment, the fabric comprises rayon. In one embodiment, the dry sanitizing patch further comprises a material comprising a plurality of plies, and one or more than one of the plurality of plies comprises the fabric. In one embodiment, the material comprises two plies. In another embodiment, the material comprises three plies. In another embodiment, the material comprises four plies.

According to one embodiment of the present invention, there is provided a method for making a dry sanitizing patch according to the present invention. The method comprises: a) providing the binding layer; b) attaching the adhesive layer to the non-contact surface of the binding layer; c) placing the backing layer onto the adhesive layer.

According to one embodiment of the present invention, there is provided a method of decreasing the transmission of one or more than one human pathogen. The method comprises: a) providing a dry sanitizing patch according to the present invention; b) removing the backing layer to expose the adhesive layer; c) attaching the dry sanitizing patch to a surface by contacting the adhesive layer with the surface; and d) contacting the skin of a person or an object with the contact surface of the binding layer so that the binding substance within the binding layer performs sanitization of the object or the skin of the person. In one embodiment, the surface is selected from the group consisting of ceramic tile, glass, granite, plastic, stone and wood. In another embodiment, the surface is selected from the group consisting of a computer keyboard, a counter top, a desk, digital recording device, a mobile phone and a motorized vehicle.

### FIGURES

These and other features, aspects and advantages of the present invention will become better understood with regard to the following description, appended claims, and accompanying figures where:

FIG. 1 is a top perspective view of a dry sanitizing patch according to one embodiment of the present invention;

FIG. 2 is a bottom perspective view of the dry sanitizing patch shown in FIG. 1;

FIG. 3 is a partial side perspective view of the dry sanitizing patch shown in FIG. 1 and FIG. 2;

FIG. 4 is a bottom perspective view of the dry sanitizing patch shown in FIG. 1, FIG. 2 and FIG. 3 with the bottom layer partially removed;

FIG. 5 is a partial top perspective view of a fabric useful in a dry sanitizing patch according to the present invention; and

FIG. 6 is a partial, cutaway, top perspective view of a material useful in a dry sanitizing patch according to the present invention, where the material comprises a fabric shown in FIG. 5.

### DESCRIPTION

According to one embodiment of the present invention, there is provided a dry sanitizing patch for use in decreasing the transmission of one or more than one human pathogen. In one embodiment, the dry sanitizing patch comprises a fabric for use in decreasing the transmission of one or more than one human pathogen. In another embodiment, the dry sanitizing patch comprises a material for use in decreasing the transmission of one or more than one human pathogen, where the material comprises a plurality of plies, and where one or more than one of the plurality of plies comprises a fabric for use in decreasing the transmission of one or more than one human pathogen. According to another embodiment of the present invention, there is provided a method for making a dry sanitizing patch for use in decreasing the transmission of one or more than one human pathogen. According to another embodiment of the present invention, there is provided a method of decreasing the transmission of one or more than one human pathogen, where the method comprises providing a dry sanitizing patch according to the present invention. The dry sanitizing patch and methods will now be disclosed in greater detail.

All dimensions specified in this disclosure are by way of example only and are not intended to be limiting. As will be understood by those with skill in the art with reference to this disclosure, the actual dimensions of any device or part of a device disclosed in this disclosure will be determined by its intended use.

Except where the context requires otherwise, the method steps disclosed are not intended to be limiting nor are they intended to indicate that each step is essential to the method or that each step must occur in the order disclosed.

As used in this disclosure, except where the context requires otherwise, the term “comprise” and variations of the term, such as “comprising,” “comprises” and “comprised” are not intended to exclude other additives, components, integers or steps.

As used in this disclosure, “human pathogen” comprises bacteria, fungi and viruses that cause human diseases, including bacteria, fungi and viruses that cause human respiratory tract infections.

As used in this disclosure, “binding substance” means a chemical group that chemically binds a human pathogen, rather than presenting only a physical barrier to spatial passage of the human pathogen. Similarly, “bind,” and its related terms such as “binds,” “binding” and “binding action,” refer to a chemical process, not merely the presentation of only a physical barrier to the spatial passage of the human pathogen.

As used in this disclosure, “dry” when used in this disclosure in conjunction with “dry sanitizing patch” means that the sanitizing patch does not have a gel or liquid impregnated within the sanitizing patch to perform sanitization but instead relies on the binding substance within the binding layer to perform sanitization.

As used in this disclosure, “cellulosic” means “comprising cellulose.”

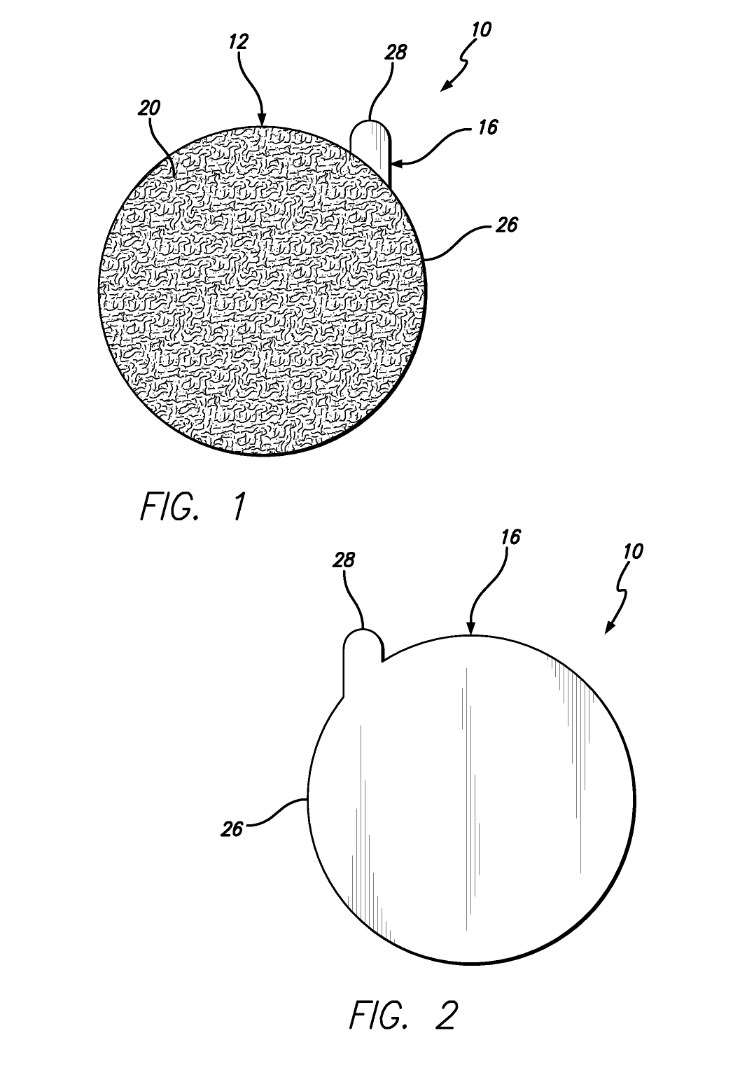

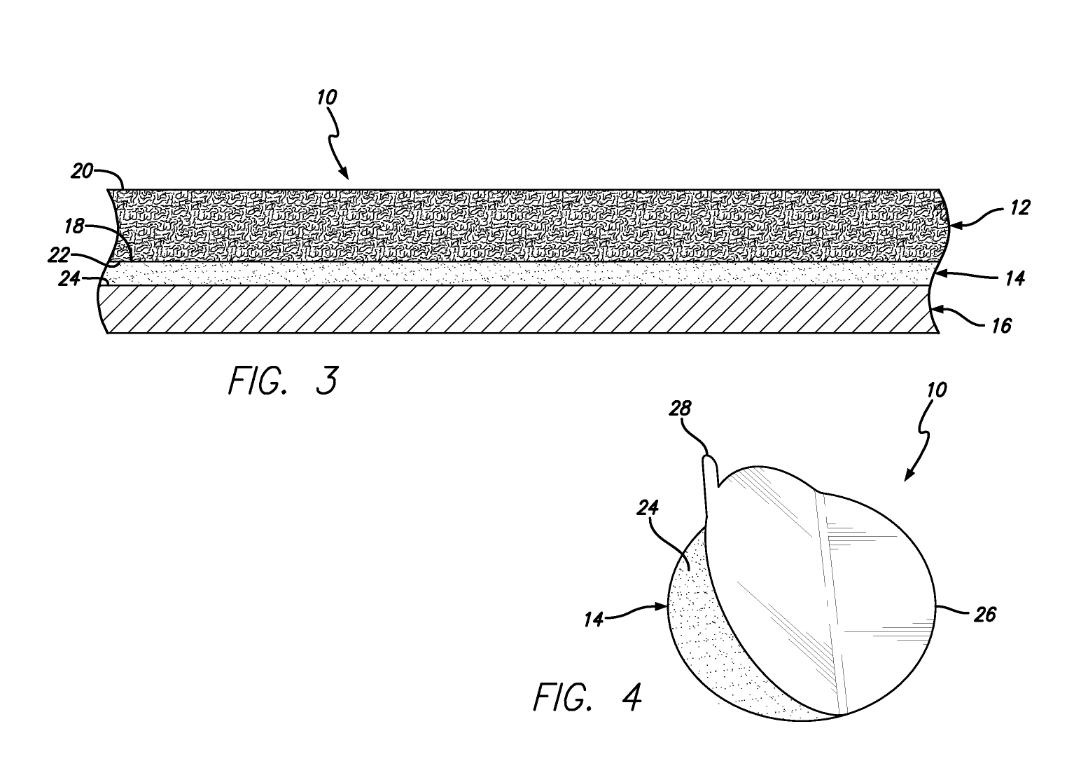

According to another embodiment of the present invention, there is provided a dry sanitizing patch for use in decreasing the transmission of one or more than one human pathogen, such as for example one or more than one virus that causes human respiratory tract infections. Referring now to FIG. 1, FIG. 2, FIG. 3 and FIG. 4, there are shown, respectively, a top perspective view of a dry sanitizing patch according to one embodiment of the present invention (FIG. 1); a bottom perspective view of the dry sanitizing patch shown in FIG. 1 (FIG. 2); a partial side perspective view of the dry sanitizing patch shown in FIG. 1 and FIG. 2 (FIG. 3); and a bottom perspective view of the dry sanitizing patch shown in FIG. 1, FIG. 2 and FIG. 3 with the bottom layer partially removed (FIG. 4). As can be seen, the dry sanitizing patch 10 comprises a plurality of layers. In one embodiment, as shown in FIG. 1, FIG. 2, FIG. 3, and FIG. 4, the dry sanitizing patch comprises three layers: a binding layer 12 comprising a fabric comprising one or more than one binding substance comprising one or more than one human pathogen binding group for chemically attaching the human pathogen to the binding substance, such as for example one or more than one type of virus that causes human respiratory tract infections; an adhesive layer 14 connected to the binding layer 12, and comprising an adhesive suitable for reversibly attaching the dry sanitizing patch to a surface; and a backing layer 16 removably connected to the adhesive layer 14 to protect the adhesive layer until the patch is ready for use. The binding layer 12 comprises a non-contact surface 18 connected to the adhesive layer 14, and comprises an opposing contact surface 20 for sanitizing the skin of a person or an object that is contacted with the contact surface 20 of the binding layer 12. The adhesive layer 14 comprises a first surface 22 connected to the non-contact surface 18 of the binding layer 12 and a second surface 24 adjacent to the backing layer 16.

The dry sanitizing patch 10 further comprises a shape defined by a perimeter 26. In one embodiment, the shape defined by the perimeter 26 is selected from the group consisting of a circle, an oval, a rectangle, a square and a triangle, though the shape defined by the perimeter 26 can be any shape suitable for the intended use of the dry sanitizing patch 10 as will be understood by those with skill in the art with reference to this disclosure. In a preferred embodiment, as shown in FIG. 1, FIG. 2 and FIG. 4, the shape defined by the perimeter 26 is a circle with a tab extension 28 defined only by the backing layer 16.

In one embodiment, the binding layer 12 of the dry sanitizing patch 10 has a surface area defined by the perimeter of between 2 cm² and 100 cm². In a preferred embodiment, the binding layer 12 of the dry sanitizing patch 10 has a surface area defined by the perimeter of between 2 cm² and 10 cm². In a preferred embodiment, the binding layer 12 of the dry sanitizing patch 10 has a surface area defined by the perimeter of between 4 cm² and 8 cm².

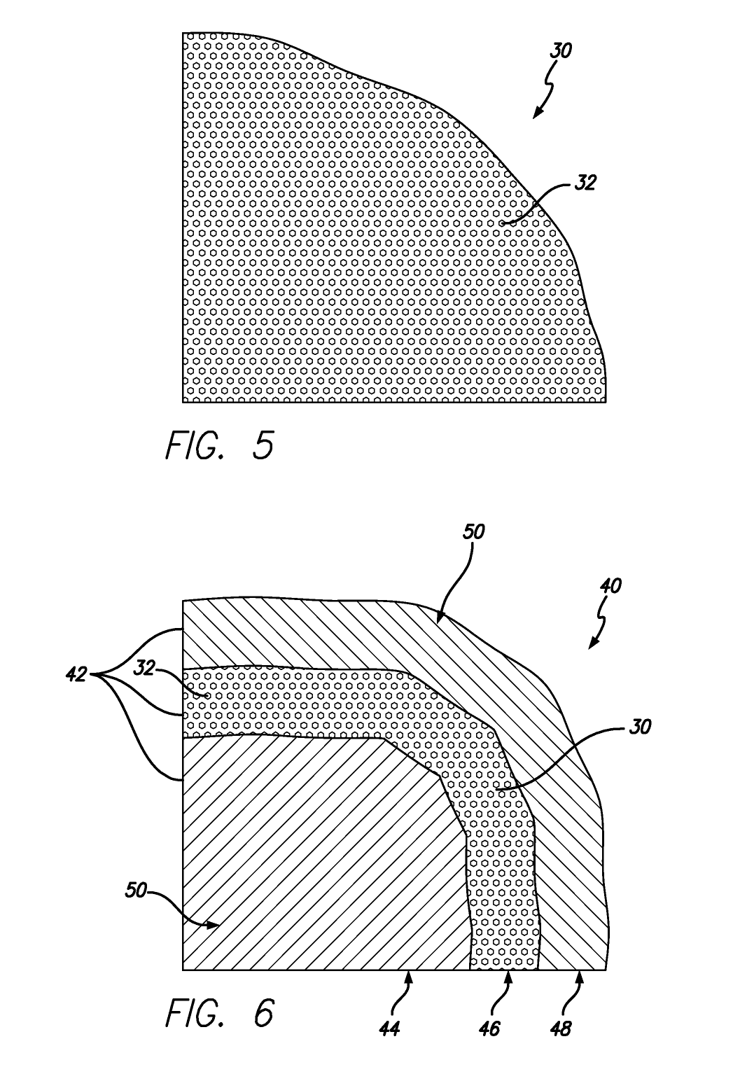

The binding layer 12 comprises a fabric for use in decreasing the transmission of human pathogens. Referring now to FIG. 5, there is shown is a partial top perspective view of a fabric useful in a dry sanitizing patch according to the present invention. As can be seen, in one embodiment, the fabric 30 comprises one or more than one binding substance 32 that binds one or more than one type of human pathogen. In a preferred embodiment, the fabric 30 comprises one or more than one binding substance 32 that binds one or more than one type of virus that causes a human disease, such as influenza virus, that causes human respiratory tract infections such as influenza. By binding the human pathogen to the fabric 30, the fabric 30 decreases the transmission of the human pathogen, such as for example by preventing release of virus particles when virus-laden droplets evaporate within the fabric 30.

The one or more than one binding substance 32 comprises one or more than one human pathogen binding group for chemically attaching the human pathogen to the binding substance 32, as will be understood by those with skill in the art with reference to this disclosure. In a preferred embodiment, the binding substance further comprises a linker group (such as for example a vinyl sulfone group) for attaching the binding substance to the fabric.

By way of example, in one embodiment, the human pathogen to be bound to the fabric 30 is selected from the group consisting of adeno-associated virus (AAV), herpes simplex virus (HSV), human papillomavirus (HPV), influenza viruses, rabies virus, respiratory syncytial virus (RSV), and the human pathogen binding group is a sialic acid group because these virus particles bind to human cells through a terminal sialic acid group on a surface oligosaccharide of the cell membrane of human cells. Sialic acid groups are, however, relatively expensive to produce in a form suitable for attachment to fibers or fabrics, and therefore, in a preferred embodiment, the binding substance 32 is a substance that mimics the binding action of sialic acid groups on influenza viruses, but that is cost effective as a component for industrial-scale production of fabrics comprising the binding substance according to the present invention.

According to one embodiment of the present invention, the one or more than one binding substance 32 comprises a human pathogen binding group selected from the group consisting of a sulfate group (such as, for example, sulfated monosaccharide or sulfated oligosaccharide) and a sulfonate group (such as, for example, sulfonated monosaccharide or sulfonated oligosaccharide), because both sulfate groups and sulfonate groups mimic the binding action of sialic acid groups on adeno-associated virus (AAV), herpes simplex virus (HSV), human papillomavirus (HPV), influenza viruses, rabies virus, respiratory syncytial virus (RSV), as well as other human pathogens, and sulfate groups and sulfonate groups can be directly linked to free hydroxyl groups and free amino groups on fibers or fabrics in a cost-effective manner for industrial-scale production in fabrics according to the present invention. In a preferred embodiment, the fabric 30 is a cellulosic fabric (i.e., comprises cellulose) and the one or more than one binding substance 32 comprises a human pathogen binding group comprising a sulfate group, yielding a fabric 30 comprising a non-hydrogel cellulose sulfate.

According to another embodiment of the present invention, the human pathogen binding group is one or more than one reactive dye comprising one or more than one sulfonate group. In a preferred embodiment, the fabric 30 is a cellulosic fabric (i.e., comprises cellulose) and the binding substance 32 is one or more than one reactive dye comprising a binding substance 32 comprising a sulfonate group, yielding a fabric 30 comprising a cellulose sulfonate.

Reactive dyes are a class of substances used to dye fibers and fabrics, both cellulosic fibers and cellulosic fabrics (such as acetate, cotton and rayon), and non-cellulosic fibers and non-cellulosic fabrics (such as wool and nylon, and fabrics made from polyester or polyolefin). Reactive dyes comprise a reactive linker group, usually either a haloheterocycle or an activated double bond that, when applied to a fiber in a dye bath, forms a covalent chemical bond with a hydroxyl group on the fiber or the fabric. Reactive dyes are classified according to the category of linker group that attaches the dye to the fiber or fabric. In one embodiment, the binding substance 32 is one or more than one reactive dye selected from the group consisting of aminochlorotriazine (Procion® H), aminochlorotriazine-sulfatoethylsulfone (Sumafix Supra), aminofluorotriazine (Cibachron F), aminofluorotriazine-sulfatoethylsulfone (Cibacron C), bis(aminochlorotriazine) (Procion®H-E) bis(aminonicotinotriazine) (Kayacelon React®), chlorodifluoropyrimidine (Drimarine K), dichloroquinoxaline (Levafix® E), dichlorotriazine (Procion MX), sulfatoethylsulfone (vinyl sulfone; Remazol®), sulfatoethylsulfonamide (Remazol® D), trichloropyrimidine (Drimarine X). Reactive dyes further comprise a chromophore group, providing the specific color for the dye. The chromophore group commonly comprises a multi-ring aromatic group; however, multi-ring aromatic groups tend to decrease water solubility, so reactive dyes usually further comprise one or more sulfonate groups to increase water solubility. The sulfonate groups of reactive dyes can function as the human pathogen binding group of the binding substance of the fabrics of the present invention, while the reactive linker groups of the reactive dyes can function as the linker group of the binding substance 32.

A given dye frequently has several trade names, but the generic names (Color Index; CI) for dyes comprise the following format: [Category (acidic, basic, direct or reactive); Color; and Number]. According to one embodiment of the present invention, the one or more than one binding substance 32 is a reactive dye selected from the group consisting of CI Reactive Blue 4, CI Reactive Blue 21, CI Reactive Blue 140, CI Reactive Blue 163, CI Reactive Brown 23, CI Reactive Orange 4, CI Reactive Red 1, CI Reactive Red 2, CI Reactive Red 6, CI Reactive Red 11, CI Reactive Red 78, CI Reactive Yellow 39, and CI Reactive Yellow 86, each of which comprises sulfonate groups which function as the human pathogen binding group suitable for binding one or more than one human pathogen according to the present invention, and each of which further comprises a linker group suitable for attaching the binding substance (the dye) to the fabric 30. In a particularly preferred embodiment, the binding substance 32 is CI Reactive Blue 21 [copper, (29H,31H-phthalocyaninato(2-)-N 29,N 30,N 31,N 32)-, sulfo((4-((2-(sulfooxy)ethyl)sulfonyl) phenyl)amino)sulfonyl derivatives] (CAS Reg. No. 73049-92-0), a sulfonated copper phthalocyanine dye with a vinyl sulfone linker group that attaches the dye to fibers and fabrics, including cellulosic fibers and fabrics. The appropriate reaction conditions for attaching reactive dyes, including for attaching CI Reactive Blue 21, to fibers and fabrics are well known to those with skill in the art, and can be found in instructions from the dye manufacturers, as well as in standard textile references, as will be understood by those with skill in the art with reference to this disclosure.

In one embodiment, the human pathogen binding group is a sulfate group that does not form a cellulose sulfate hydrogel within the fabric 30. Using a reactive dye as the binding substance 32 in the fabric 30 according to the present invention is particularly advantageous because the amount of reactive dye binding to a fabric 30 is never high enough to cause the sulfonate groups in the reactive dyes to make a hydrogel in the fabric 30.

As will be understood by those with skill in the art with reference to this disclosure, both cellulose sulfate and cellulose sulfonate have surfactant properties, so that fabrics comprising cellulose sulfate or cellulose sulfonate disrupt virus-laden droplets and exposes the virus particles to the sulfate groups on the cellulose sulfate, and to the sulfonate groups on the cellulose sulfonate, thereby trapping the virus particles within the fabric 30.

In one embodiment, the fabric 30 of the present invention further comprises one or more than one additional substance, other than the binding substance 32 and the fibers of the fabric 30, that decreases the pathogenic capacity of one or more than one human pathogen. In a preferred embodiment, the one or more than one additional substance is one or more than one type of multivalent metallic ion, such as for example multivalent copper, multivalent silver or multivalent zinc, all of which are viricidal, bactericidal and fungicidal. In a preferred embodiment, the metallic salt is a divalent metallic salt. In another embodiment, the one or more than one substance is a metallic salt, such as for example copper acetate, copper oxide, copper sulfate or zinc acetate, all of which are bactericidal, viricidal and fungicidal.

As will be understood by those with skill in the art with reference to this disclosure, using a binding substance 32 comprising a sulfate group or a sulfonate group on a fabric 30 comprising cellulose is both relatively inexpensive and suitable for industrial-scale production of dry sanitizing patches 10 according to the present invention. Further, the fabric 30 according to the present invention is safe to both people and pets because the fabric 30 is not toxic and because binding the virus particles within the fabric 30 prevents the virus particles from leaching out of the fabric 30 after the virus particles contact the fabric 30. Further advantageously, the fabric 30 of the present invention does not require illumination and singlet oxygen generation for decreasing the transmission of one or more than one human pathogen, as with some fabrics designed to decrease transmission of one or more than one human pathogen.

In one embodiment, the fabric 30 is woven, such as, for example, woven rayon.

In another embodiment, the fabric 30 is non-woven, such as, for example, non-woven rayon.

In one embodiment of the present invention, the binding layer 12 of the dry sanitizing patch 10 comprises a material for use in decreasing the transmission of one or more than one human pathogen. Referring now to FIG. 6, there is shown is a partial, cutaway, top perspective view of a material useful in a dry sanitizing patch according to the present invention, where the material comprises a fabric shown in FIG. 5. As can be seen, the material 40 comprises a plurality of plies 42, and where one or more than one of the plurality of plies 42 comprises a fabric 30 according to the present invention. The material 40 can comprise two plies, three plies (as shown), four plies or more than four plies, as will be understood by those with skill in the art with reference to this disclosure. In a preferred embodiment, the plurality of plies 42 is two plies. In a particularly preferred embodiment, the plurality of plies 42 is three plies 44, 46 and 48 (as shown). In another particularly preferred embodiment, the plurality of plies 42 is four plies.

At least one of the plies 42 of the material 40 comprises a fabric 30 (here shown as ply 46) according to the present invention. In a preferred embodiment, one or more than one of the plies 42 of the material 40 is a heat-moldable fabric 50, such as a heat-moldable fabric 50 selected from the group consisting of polypropylene, polyester and non-woven cellulose acetate fabric 30. In one embodiment, the heat-moldable fabric 50 comprises polypropylene webbing which traps airborne particles, but is relatively water repellent so that virus-laden droplets are normally not disrupted even if the virus-laden droplets are trapped within the webbing.

A method of making the fabric 30 used in the dry sanitizing patch 10 will now be disclosed by way of example only primarily with respect to making a fabric 30 comprising cellulose (in this example, rayon) with binding substances 12 comprising sulfate groups as the human pathogen binding group, though other methods can be used to produce the same fabric 30, and corresponding fabrics with other binding substances (such as sulfonate groups) according to the present invention, as will be understood by those with skill in the art with reference to this disclosure.

In one embodiment, the method comprises, first, providing fibers suitable for use in a fabric for decreasing the transmission of one or more than one human pathogen. In one embodiment, the fabric comprises cellulose. In a preferred embodiment, the fabric comprises rayon (a form of cellulose). The most important source of cellulose fibers for commercial purposes is from wood pulp; however, cellulose fibers obtained directly from wood pulp are too short and coarse to weave into a fabric according to the present invention, and cellulose derived from wood pulp is relatively insoluble in organic solvents and cannot be extruded into fine fibers. By contrast, rayon fibers are produced from naturally occurring cellulose polymers derived from wood pulp and other plants. To form rayon fibers, the cellulose is first derivatized with solubilizing groups (such as for example acetate), formed into spun fibers, and then, the solubilizing groups are removed yielding cellulose fibers that can be woven into fabric, as will be understood by those with skill in the art with reference to this disclosure.

Next, the method comprises adding one or more than one binding substance to the fibers. Adding the binding substance to the fibers can be accomplished using techniques known to those with skill in the art, as will be understood by those with skill in the art with reference to this disclosure. In a preferred embodiment, the binding substance added is a binding substance according to the present invention. By way of example, the method will be disclosed with respect to binding substances comprising a human pathogen binding group that comprises a sulfate group, thereby yielding sulfated cellulose fibers. In this embodiment, adding one or more than one binding substance to the fibers results in sulfation of the cellulose derived fibers in the fabric without disrupting the structure or strength of the fabric. Further, though these steps are disclosed with respect to covalently bonding sulfate groups to cellulosic fibers (such as rayon), equivalent steps can be used for adding sulfate groups to other cellulosic fabrics, blends of cellulose-derived and noncellulose-derived fibers (such as for example fibers made from polyester or polyolefin) and noncellulose-derived fibers that comprise free hydroxyl or amino groups, as will be understood by those with skill in the art with reference to this disclosure.

Cellulose is a linear polymer of glucose units, each of which has three free hydroxyl groups. The degree of sulfation (DS) of cellulose is defined in the art as the average number of sulfate groups per monosaccharide unit. A DS of 3 is the maximum possible, indicating that all available hydroxyl groups are fully sulfated. A degree of sulfation of 1 indicates that an average of one sulfate group per glucose unit is present, and a DS of 0.1, for example, indicates that an average of one hydroxyl group of every ten glucose units is sulfated. An important aspect of the present invention is that the binding of viruses and other human pathogens to a fiber or fabric according to the present invention involves binding of the human pathogen to more than one immobilized sulfate group or sulfonate group on the fiber or fabric, thereby strongly increasing the affinity of the interaction between the binding substance and the human pathogen.

The degree of sulfation is determined by any suitable analytical method that measures sulfate, sulfonate or total sulfur, such as for example by elemental analysis. The sulfur content of cellulose fibers without a binding substance attached or nonpigmented cellulose fibers or fabrics is extremely low or undetectable. According to one embodiment of the present invention, the present method results in a degree of sulfation between 0.02 and 2. In a preferred embodiment of the present invention, the present method results in a degree of sulfation between 0.05 and 0.5. In a particularly preferred embodiment, the present method results in a degree of sulfation of between 0.09 and 0.21. The degree of sulfation for sulfated or sulfonated fibers or fabric can be regulated by adjusting the time, temperature or reagent concentrations in a sulfation or sulfonation reaction, as will be understood by those with skill in the art with reference to this disclosure, to produce fibers with the required degree of sulfation.

As the degree of sulfation increases above 0.2 for a cellulosic fabric, the water solubility of fibers increases when exposed to liquid water or water vapor, causing the fabric to form a hydrogel and decrease gas permeability through the fabric. In a preferred embodiment, the method further comprises crosslinking the fibers of the fabric, before or after attaching the binding substance, by treating the fabric with one or more than one crosslinking agent that chemically bonds the fibers of the fabric to one another thereby preventing solubilization. In one embodiment, treating the fabric with a crosslinking agent comprises contacting the fabric with an alkali, e.g., sodium hydroxide, to give the alkalinized cellulose in the case of cellulosic fabrics, and then reacting the fabric with the crosslinking agent. In one embodiment, the crosslinking agent is selected from the group consisting of dichloroalkanes, dimethylolureas, formaldehyde and trimethylol-melamines In a preferred embodiment, the crosslinking agent is an epoxy compound selected from the group consisting of diethylene glycol diglycidyl ether, ethylene glycol diglycidyl ether, epichlorohydrin, glycerin diglycidyl ether and vinylcyclohexene dioxide.

Adding one or more than one binding substance comprising a sulfate human pathogen binding group to the fibers can be accomplished, for example, by first, contacting the fabric with a suitable solvent, such as for example dimethylsulfoxide (DMSO) or dimethylformamide (DMF). The amount of time that the fabric is contacted with the solvent is adjusted to optimize fiber swelling, thereby increasing exposure of hydroxyl groups on the fiber surface to sulfation, as will be understood by those with skill in the art with reference to this disclosure.

Next, the solvent treated fabric is contacted with the binding substance, such as for example a sulfating reagent. Suitable sulfating reagents depend on the solvent used, as will be understood by those with skill in the art with reference to this disclosure. For example, in one embodiment, the solvent is dimethylsulfoxide, and the sulfating reagent is DMSO treated with sulfur trioxide (DMSO—SO₃). In another embodiment, the solvent is dimethylformamide, and the sulfating reagent is dimethylformamide treated with sulfur trioxide (DMF-SO₃). Contact with the binding substance is maintained until a satisfactory degree of covalent binding of the binding substance to the fibers is achieved but before excess binding substance binds to the fibers, which in the case of sulfate would render the fabric impermeable to gas upon contact with liquid water or water vapor, as will be understood by those with skill in the art with reference to this disclosure.

In one embodiment, the method further comprises rinsing the fabric with a solvent, such as for example (DMSO—SO₃) and (DMF-SO₃) and then contacting the fabric with a suitable base, such as for example sodium hydroxide, sodium acetate, or sodium bicarbonate, to neutralize an acidic binding substance such as an acidic sulfating agent, or to neutralize acid formed during the addition of the binding substance to the fabric.

The fabric is then washed with a suitable solvent, such as for example water or a simple alcohol (ethanol or isopropanol) to remove unreacted reagents yielding the sulfated fabric suitable for use in decreasing the transmission of one or more than one human pathogen, including viruses that cause human respiratory tract infections.

In another embodiment, the method of the present invention for making a fabric for use in decreasing the transmission of one or more than one human pathogen comprises, first, providing cellulose sulfate material made from cellulose pulp or cellulose powder and having a degree of sulfation greater than 0.2, and preferably greater than 0.5 sufficient to render the fibers water soluble. Next, the soluble cellulose sulfate is then applied to a fabric and covalently linked to the fibers of the fabric with a crosslinking agent, as disclosed above, as will be understood by those with skill in the art with reference to this disclosure. In this embodiment of the method, the fabric is not exposed to the relatively harsh sulfation conditions and reagents, but only to soluble cellulose sulfate and to the crosslinking reagents, and to the conditions for crosslinking, thereby reducing the potential for damage to the fabric that can occur if the sulfation reaction is not well controlled. A concentration of soluble cellulose sulfate is selected by testing, as will be understood by those with skill in the art with reference to this disclosure.

In one embodiment of the present invention, the method further comprises contacting the fabric with one or more than one substance that chemically disrupts a characteristic of the human pathogen essential for human pathogenicity. In a preferred embodiment, the one or more than one substance is a multivalent metallic ion, such as for example multivalent copper, multivalent silver or multivalent zinc, all of which are viricidal, bactericidal and fungicidal. In another embodiment, the one or more than one substance is a metallic salt, such as for example copper oxide, zinc acetate, copper acetate or copper sulfate, all of which are bactericidal, viricidal and fungicidal. In a preferred embodiment, the metallic salt is a divalent metallic salt. Acetate is advantageous as an anionic salt constituent as it is volatile and can be removed from the fabric by evaporation, but other anions are also suitable as salt components, including chlorides, oxides, iodides and others. The addition of the one or more than one substance to the fabric increases the effectiveness of the dry sanitizing patch of the present invention in decreasing the transmission of one or more than one human pathogen by using mechanisms in addition to binding the human pathogen to the fabric.

In one embodiment of the present invention, the method further comprises incorporating one or more than one type of fiber other than the fibers comprising the binding substance, such as for example polyester fibers or polypropylene fibers, into the fabric.

In another embodiment, cellulosic fibers in the form of staple or tow are sulfated by the same types of sulfation reactions used for fabrics as disclosed in this disclosure, and then the cellulose sulfate fibers are washed and then formed into a nonwoven or woven fabric by conventional methods whereby cellulosic staple or tow are spun into threads or directly formed into nonwoven fabrics.

The method of the present invention for making a fabric for use in decreasing the transmission of one or more than one human pathogen, will now be disclosed with respect to the following examples.

### Example 1

### Preparation of Sulfated Rayon Fabric

According to one embodiment of the present invention, sulfated rayon was prepared according to the present invention as follows. First, 60 ml isopropanol was chilled on ice and 0.2 grams MgSO₄ was added to the isopropanol to remove water. Next, 240 ml sulfuric acid, previously chilled on ice, was added to the isopropanol. Then, nonwoven rayon fabric having a density of 70 grams/meter² was cut into 17.5 cm by 22.5 cm rectangles and laid on polypropylene mesh of approximately the same size. Next, the rayon fabric on the mesh was submerged in chilled acetic acid for 15 minutes. Then, the isopropanol/sulfuric acid mixture was poured into a polyethylene box (approximately 30 cm by 37.5 cm) sitting on ice. Next, the rayon fabric on the polyethylene mesh was submerged in the isopropanol/sulfuric acid mixture for either 5 minutes or for 10 minutes, and rinsed first in cold isopropanol, and then in cold isopropanol containing 3 grams of sodium acetate per 100 ml, and then in cold isopropanol producing the sulfated rayon fabric. Next, the rayon fabric was then allowed to dry while still on the polyethylene mesh. Samples of the sulfated rayon fabric were analyzed for sulfur and carbon content. A 5 minute reaction time prior to rinsing was found to yield a degree of sulfation (DS) of approximately 0.1, while a 10 minute reaction time prior to rinsing was found to yield a degree of sulfation (DS) of approximately 0.2.

### Example 2

### Preparation of Sulfonated Rayon Fabric

According to one embodiment of the present invention, sulfonated rayon fabric was prepared according to the present invention as follows. First, a solution was prepared by adding 30 grams of sodium sulfate to 600 grams distilled water, followed by adding of 4 grams of CI Reactive Blue 21 dye (a sulfonated binding substance). Next, 30 grams of nonwoven rayon fabric having a density of 70 grams/meter² were added to the solution and gently swirled until uniformly submerged and wetted. Then, 12 grams of sodium carbonate were added with stirring, and the mixture was held at 30° C. for 35 minutes. Next, the temperature was raised to 70° C. for an additional 60 minutes yielding the sulfonated rayon fabric (with CI Reactive Blue 21 dye as the binding substance). Then, the sulfonated rayon fabric was rinsed under running water until no more free dye was eluted, and the sulfonated rayon fabric was air-dried.

### Example 3

### Preparation of Fabric Comprising One or More than One Substance that Destroys the Pathogenic Capacity of One or More than One Human Pathogen

According to one embodiment of the present invention, sulfated cellulose fabric made according to Example 1 or sulfonated cellulose fabric made according to Example 2 was prepared to comprise one or more than one than one additional substance, other than the binding substance, that destroys the pathogenic capacity of one or more than one human pathogen as follows. First, sulfated rayon fabric was made according to the process disclosed in Example 1, or sulfonated rayon fabric (with CI Reactive Blue 21 dye as the binding substance) was made according to the process disclosed in Example 2. Then, copper sulfate and zinc acetate, both of which are divalent metal salts, were applied by aerosol to the fabric at 40 μl/cm² fabric using a concentration of 1 gram metal salt/100 milliliters of water. The fabric comprising the additional substance was then air-dried yielding sulfated rayon fabric comprising both divalent copper and divalent zinc ions, or sulfonated rayon fabric comprising both divalent copper and divalent zinc ions.

### Example 4

### Industrial Process for Preparation of Sulfonated Rayon Fabric Comprising Divalent Metal Salts

According to one embodiment of the present invention, sulfonated rayon fabric (with CI Reactive Blue 21 dye as the binding substance) and comprising divalent metal salts was prepared according to the present invention as follows. First, 100% spunlace viscose rayon fabric having a density of 70 grams/meter² was dyed with CI Reactive Blue 21 (Novacron® Turquoise H-GN) at a liquid to solid ratio of 20:1. Next, 50 g/L sodium sulfate, 20 g/L sodium carbonate and 12% dye by volume (120 ml/L) was added to a dye bath and mixed thoroughly with continuous agitation. Then, the rayon fabric was immersed in the dye bath for 35 minutes at a temperature of 30° C., followed by 60 minutes at a temperature of 70° C. producing the sulfonated rayon fabric (with CI Reactive Blue 21 dye as the binding substance). Next, the sulfonated rayon fabric was rinsed under running water and air-dried. Then, 50 grams each of copper acetate and zinc acetate per liter of water was sprayed on the sulfonated rayon at rate 0.08 L/m² producing the sulfonated rayon fabric comprising both divalent copper and divalent zinc ions. The sulfonated rayon fabric comprising both divalent copper and divalent zinc ions was again air-dried.

### Example 5

### Assessment of Fabric for Anti-Human Pathogen Properties

Testing antiviral properties (as a surrogate for anti-human pathogen properties) of a fabric is performed by application of standardized amounts of a virus onto a piece of test fabric. The test fabric is then stirred in cell culture medium to elute any functional virus particles, that is, virus particles that are not inactivated by adherence to the fabric or otherwise to the test fabric. Functional virus particles eluted into the culture medium are assayed for viral activity by contacting the medium with cells susceptible to viral killing, and ascertaining a quantitative readout of cell death. Decreased cell death in the eluting medium indicates increased inactivation of the virus by the test fabric through viral adherence to the fabric or otherwise by the test fabric.

According to one embodiment of the present invention, sulfated rayon fabric having a degree of sulfation (DS) of 0.2, made according to Example 1, sulfonated rayon fabric (with CI Reactive Blue 21 dye as the binding substance), made according to Example 2, and sulfonated rayon fabric (with CI Reactive Blue 21 dye as the binding substance) comprising both copper sulfate and zinc acetate, made according to Example 3, were assessed for antiviral properties. First, test samples of the sulfated rayon fabric, the sulfonated rayon fabric (with CI Reactive Blue 21 dye as the binding substance), and the sulfonated rayon fabric (with CI Reactive Blue 21 dye as the binding substance) comprising both copper sulfate and zinc acetate were submitted to Microbiotest, Inc. (Sterling, Va. US) for assessment of the fabric's ability to inactivate the human pathogen herpes simplex virus (HSV). HSV was applied in an aerosol to a 5 cm by 5 cm area of the test fabrics, as well as to a non-sulfated, non-sulfonated piece of rayon control fabric, and to a piece of rayon fabric treated only with copper sulfate and zinc acetate (1 gram each per 100 ml water, applied at 40 microliters per square centimeter). The HSV-treated fabric samples were held for 1 minute and then placed in individual 20 ml aliquots of extraction medium and subjected to gentle agitation for 5 minutes. Aliquots of the extraction sample were serially diluted 10-fold in dilution medium and inoculated onto host cells. Residual infectious virus in extraction medium from each sample was detected and quantified by their viral-induced cytopathic effects.

TABLE 1
RESULTS OF ASSESSMENT OF FABRIC FOR
ANTIVIRAL PROPERTIES

| | LOGS OF |
|---|---|
| | INFECTIOUS HSV |
| | RECOVERED AFTER |
| | 1 MINUTE VIRUS |
| | CONTACT TIME |
| FABRIC TESTED | WITH THE FABRIC |
| non-sulfated, non-sulfonated rayon control | 7.60 ± 0.19 |
| fabric | |
| non-sulfated, non-sulfonated rayon fabric | 5.60 ± 0.23 |
| treated with copper sulfate and zinc acetate | |
| sulfated rayon fabric | 5.73 ± 0.24 |
| sulfonated rayon fabric (with CI Reactive | 7.23 |
| Blue 21 dye as the binding substance) | |
| sulfonated rayon fabric (with CI Reactive | undetectable (below 3.13) |
| Blue 21 dye as the binding substance) | |
| comprising copper sulfate and zinc acetate | |

As can be seen, the sulfated rayon fabric prepared according to Example 1, had a 1.87 log reduction in pathogenic virus as compared to the non-sulfated, non-sulfonated rayon control fabric. Incorporating copper sulfate and zinc acetate to the non-sulfated, non-sulfonated rayon control fabric yielded a 2.0 log reduction in pathogenic virus as compared to the non-sulfated, non-sulfonated rayon control fabric, where the reduction in pathogenic virus was attributable to the presence of the divalent salts alone. Sulfonated rayon fabric (with CI Reactive Blue 21 dye as the binding substance) prepared according to Example 2 had a 0.37 log reduction in pathogenic virus as compared to the non-sulfated rayon control fabric.

The lower limit of detection in the assay system was 3.13 logs, so that the minimum reduction in HSV titer for sulfonated rayon fabric (with CI Reactive Blue 21 dye as the binding substance) and treated with copper sulfate and zinc acetate was 4.47 logs. Thus, a minimum of 2.47 logs further viral inactivation or trapping was achieved with sulfonation and divalent metal ions versus non-sulfated, non-sulfonated rayon fabric incorporating the same amount of divalent metal ions. These results demonstrate an unexpected synergy with respect to anti-human pathogen activity between sulfonation of a fabric and the incorporation of divalent metal salts into the fabric.

According to one embodiment of the present invention, sulfated rayon fabric having a degree of sulfation (DS) of either 0.1 or 0.2 made according to Example 1, sulfated rayon fabric having a degree of sulfation (DS) of 0.2 and comprising the divalent metal salts copper sulfate and zinc acetate made according to Example 4, sulfonated rayon fabric (with CI Reactive Blue 21 dye as the binding substance) made according to Example 2, and sulfonated rayon fabric (with CI Reactive Blue 21 dye as the binding substance) and comprising the divalent metal salts copper sulfate and zinc acetate made according to Example 3, as well as to a non-sulfated, non-sulfonated rayon control fabric, and rayon fabric comprising the divalent metal salts copper sulfate and zinc acetate were assessed for their antiviral properties. 4.70 logs of influenza A virus was applied in an aerosol to a 5 cm by 5 cm area of the test fabrics and three samples of each of the test fabrics with the applied influenza A virus was allowed to sit after virus application for either 1, 5, or 15 minutes, and then placed in individual 20 ml aliquots of extraction medium and subjected to gentle agitation for 5 minutes. Serial dilutions of extraction buffers were administered into embryonated eggs for assay of pathogenic influenza A viral titer by embryonic viability and by a hemaglutinin assay of allantoic fluid from such eggs.

The results of the testing were that the sulfated rayon fabric made according to Example 1 having a degree of sulfation (DS) of either 0.1 or 0.2, both yielded no detectable pathogenic virus at each of the time points tested (1, 5 and 15 minutes), indicating an influenza virus log reduction greater than 3 at each of the time points tested compared to the amount of virus applied to the fabric. Similarly, sulfated rayon fabric made according to Example 1 having a degree of sulfation (DS) of 0.2 and comprising the divalent metal salts copper sulfate and zinc acetate also yielded no detectible pathogenic virus at each of the time points tested (1, 5 and 15 minutes), indicating an influenza virus log reduction greater than 3 at each of the time points tested compared to the amount of virus applied to the fabric.

Sulfonated rayon fabric (with CI Reactive Blue 21 dye as the binding substance), made according to Example 2, reduced influenza A virus in log reductions of 1.95 at a 1 minute test time, 2.33 at a 5 minute test time, and 3.08 at 15 minute test time. Sulfonated rayon fabric (with CI Reactive Blue 21 dye as the binding substance) and comprising the divalent metal salts copper sulfate and zinc acetate made according to Example 3, yielded no detectible pathogenic virus at each of the time points tested (1, 5 and 15 minutes), indicating an influenza virus log reduction greater than 3 at each of the time points tested.

According to another embodiment of the present invention, there is provided a method for making a dry sanitizing patch according to the present invention for use in decreasing the transmission of one or more than one human pathogen, including viruses that cause human respiratory tract infections. In one embodiment, the method comprises, first providing a binding layer comprising a contact surface and an opposing non-contact surface, where the binding layer comprises a fabric comprising one or more than one binding substance; where the one or more than one binding substance comprises one or more than one human pathogen binding group for chemically attaching the human pathogen to the binding substance according to the present invention. Next, an adhesive layer is attached to the non-contact surface of the binding layer. Then, a backing layer is placed onto the adhesive layer.

In one embodiment, the binding layer comprises a material according to the present invention, where the material comprises a plurality of plies and one of the plurality of plies comprises a fabric comprising one or more than one binding substance; where the one or more than one binding substance comprises one or more than one human pathogen binding group for chemically attaching the human pathogen to the binding substance according to the present invention.

According to another embodiment of the present invention, there is provided a method of decreasing the transmission of one or more than one human pathogen. In one embodiment, the method comprises, first, providing a dry sanitizing patch according to the present invention. Next, the backing layer is removed to expose the adhesive layer. Then, the dry sanitizing patch is attached to a surface by contacting the adhesive layer with the surface. In one embodiment, the surface is selected from the group consisting of ceramic tile, glass, granite, plastic, stone and wood, such as for example a surface of a computer keyboard, a counter top, a desk, digital recording device, a mobile phone or a motorized vehicle. Next, the skin of a person or an object is contacted with the contact surface of the binding layer so that the binding substance within the binding layer performs sanitization of the object or the skin of the person.

Although the present invention has been discussed in considerable detail with reference to certain preferred embodiments, other embodiments are possible. Therefore, the scope of the appended claims should not be limited to the description of preferred embodiments contained in this disclosure.

What is claimed is:

1. A dry sanitizing patch for use in decreasing the transmission of at least one human pathogen, said dry sanitizing patch comprising:
a binding layer comprising a fabric incorporating both multivalent copper and multivalent zinc, and at least one binding substance, wherein said binding substance includes at least one human pathogen binding group capable of attaching to said human pathogen, wherein said binding substance is CI Reactive Blue 21 and wherein said human pathogen binding group is selected from the group consisting of one or both of a sulfate group and a sulfonate group, in such concentration to result in said fabric having a degree of sulfation between 0.02 and 0.21;
said binding layer having a contact surface and a non-contact surface;
an adhesive layer attached to said non-contact surface; and
a backing layer removably connected to said adhesive layer to protect said adhesive layer until said patch is ready for use.

2. A dry sanitizing patch for use in decreasing the transmission of at least one human pathogen, said dry sanitizing patch comprising:
a binding layer comprising a fabric incorporating at least one type of multivalent metallic ion or metallic salt, and at least than one binding substance, wherein said binding substance includes at least one human pathogen binding group capable of attaching to said human pathogen, wherein said binding substance is at least one reactive dye and wherein said human pathogen binding group is selected from the group consisting of one or both of a sulfate group and a sulfonate group, in such concentration to result in said fabric having a degree of sulfation between 0.02 and 0.21;
said binding layer having a contact surface and a non-contact surface;
an adhesive layer attached to said non-contact surface; and
a backing layer removably connected to the said adhesive layer to protect said adhesive layer until said patch is ready for use.

3. (canceled)

4. (canceled)

5. The dry sanitizing patch of claim 2, where said binding layer of said dry sanitizing patch has a surface area defined by a perimeter of between 2 cm² and 100 cm².

6. The dry sanitizing patch of claim 2, where said binding layer of said dry sanitizing patch has a surface area defined by a perimeter of between 2 cm² and 10 cm².

7. The dry sanitizing patch of claim 2, where said binding layer of said dry sanitizing patch has a surface area defined by a perimeter of between 4 cm² and 8 cm².

8. (canceled)

9. The dry sanitizing patch of claim 2, where said reactive dye is selected from the group consisting of CI Reactive Blue 4, CI Reactive Blue 21, CI Reactive Blue 140, CI Reactive Blue 163, CI Reactive Brown 23, CI Reactive Orange 4, CI Reactive Red 1, CI Reactive Red 2, CI Reactive Red 6, CI Reactive Red 11, CI Reactive Red 78, CI Reactive Yellow 39, and CI Reactive Yellow 86.

10. (canceled)

11. (canceled)

12. The dry sanitizing patch of claim 2, wherein said multivalent metallic ion is selected from the group consisting of multivalent copper, multivalent silver and multivalent zinc.

13. The dry sanitizing patch of claim 2, wherein said multivalent metallic ion is selected from the group consisting of copper acetate, copper oxide, copper sulfate and zinc acetate.

14. (canceled)

15. The dry sanitizing patch of claim 2, wherein said binding substance comprises a linker group for attaching said binding substance to said fabric.

16. The dry sanitizing patch of claim 2, wherein said fabric comprises cellulosic material.

17. The dry sanitizing patch of claim 2, further comprising a material comprising a plurality of plies, and wherein at least one of the plurality of plies comprises said fabric.

18. The dry sanitizing patch of claim 17, wherein said material comprises two plies.

19. The dry sanitizing patch of claim 17, wherein said material comprises three plies.

20. The dry sanitizing patch of claim 17, wherein said material comprises four plies.

21. A method for making said dry sanitizing patch of claim 2, the method comprising:
a) providing said binding layer;
b) attaching said adhesive layer to said non-contact surface of said binding layer; and
c) placing said backing layer onto said adhesive layer.

22. A method of decreasing the transmission of at least one human pathogen, said method comprising:
a) providing a dry sanitizing patch according to claim 1;
b) removing said backing layer to expose said adhesive layer;
c) attaching said dry sanitizing patch to a surface by contacting said said adhesive layer with said surface; and
d) contacting the skin of a person or an object with said contact surface of said binding layer so that said binding substance within said binding layer performs sanitization of said object or said skin of said person.

23. The method of claim 22, wherein said surface is selected from the group consisting of ceramic tile, glass, granite, plastic, stone and wood.

24. The method of claim 22, wherein said surface is selected from the group consisting of a computer keyboard, a counter top, a desk, digital recording device, a mobile phone and a motorized vehicle.

25. A method of decreasing the transmission of at least one human pathogen, said method comprising:
a) providing a dry sanitizing patch according to claim 2;
b) removing said backing layer to expose the adhesive layer;
c) attaching said dry sanitizing patch to a surface by contacting said adhesive layer with said surface; and
d) contacting the skin of a person or an object with said contact surface of said binding layer so that said binding substance within said binding layer performs sanitization of said object or said skin of said person.

26. The method of claim 25, wherein said surface is selected from the group consisting of ceramic tile, glass, granite, plastic, stone and wood.

27. The method of claim 25, wherein said surface is selected from the group consisting of a computer keyboard, a counter top, a desk, digital recording device, a mobile phone and a motorized vehicle.
